(19) United States
(12) Patent Application Publication
(10) Pub. No.: US 2009/0214669 A1
(43) Pub. Date: Aug. 27, 2009

(54) Combination anticancer therapy and pharmaceutical compositions therefore

(76) Inventors: Jacques Alain Bauer, Saint Prex (CH); Carlo Chiavaroli, Thoiry (FR)

Correspondence Address: WILEY REIN LLP, 1776 K. STREET N.W., WASHINGTON, DC, 20006, US

(21) Appl. No.: 11/658,384

(22) PCT Filed: Jul. 23, 2005
(86) PCT No.: PCT/IB2005/000944

§ 371 Date: Apr. 8, 2009

(30) Foreign Application Priority Data
Jul. 23, 2004 (IB) 2004/002378

(51) Int. Cl. A61K 31/7016; A61K 33/24; A61P 35/00

(52) U.S. Cl. 424/649; 514/42

(57) ABSTRACT

This invention relates to anticancer therapy and more precisely to the immunological control of cancer.

Specifically this invention relates to pharmaceutical compositions incorporating as the active ingredient at least one immuno-stimulating agent with charged or central groups of general formula I

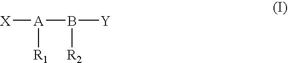

wherein X, Y, A, B, R₁ and R₂ are as defined in the specification together with a radiotherapy method suitable to fight cancer or together with a known antineoplastic chemotherapeutic agent selected from the group consisting of alkylating agents, anti-metabolic agents, agents acting on tubules and tyrosine Kinase inhibitors in conjunction or admixture with an inert non toxic pharmaceutically acceptable diluent or carrier.

This invention also relates to the salts of a compound of general formula I with a mineral or organic base, namely a pharmaceutically-acceptable base.

Use for treating cancer conditions within a single container or disposed within distinct container

This invention relates to the immunological control of cancer.

This invention relates to pharmaceutical compositions increasing or improving the efficacy of known antineoplastic agents or radiotherapy methods by stimulating the [cancer] patient's immune system.

More precisely this invention relates to pharmaceutical compositions incorporating as the active ingredients a combination of an immunostimulating agent and a known or experimental antineoplastic agent in admixture or combination with one or several diluent or excipient.

Specifically this invention also relates to a combination of an immunostimulating agent and recognized radiotherapy methods to fight cancer in admixture or combination with a carrier or vehicle intended for oral, injectable way.

More specifically, the present invention has, as a subject matter, pharmaceutical compositions combining as the active ingredients at least ones immunostimulating agent with charged or neutral groups of general formula (I):

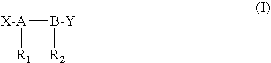

wherein
A-B is a disaccharide,
X and Y are charged or neutral functional groups,
R₁ and R₂ are hydroxyacyl groups which may be acylated with an aliphatic carboxylic acid,
together with a radiotherapy method suitable to fight cancer, or together with a known antineoplastic chemotherapeutic agent selected from the group consisting of alkylating agents, antimetabolites, agents acting on tubules, tyrosine-kinase inhibitors,
in conjugation or admixture with an inert non-toxic pharmaceutically acceptable diluent or carrier.

The invention also relates to the salts of a compound of general formula (I) with a mineral or organic base and namely a pharmaceutically acceptable base.

This invention also relates to a pharmaceutical composition wherein the immunologically-active compound is a diacylated compound with charged or neutral groups, of general formula I:

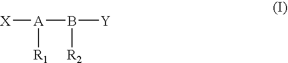

wherein
A and B is the β-(1,6) linked diglucosamine disaccharide back bone of lipid A of formula (II):

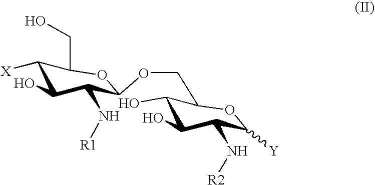

wherein
R₁ and R₂ each designate an acyl group derived from a saturated or unsaturated, straight or branched-chain carboxylic acid having from 2 to 24 carbon atoms, which is unsubstituted or bears one or more substituents selected among hydroxyl, alkyl, alkoxy, acyloxy, amino, acylamino, acylthio and alkylthio groups.
X designates a neutral or charged group selected among the following groups: dihydroxyphosphoryloxy, hydroxysulfonyloxy, hydroxyl, carboxyalkoxy, carboxyalkylthio, carboxyacyloxy, carboxyaminoacyloxy, or diaminoacyloxy and aminoacyloxy and the wavy line indicates an a or C configuration
Y designates a neutral or charged group selected among the following groups: dihydroxyphosphoryloxy, hydroxysulfonyloxy, hydroxyle, carboxyalkoxy, carboxyalkylthio, carboxyaminoalkoxy and aminoalkoxy.

in combination with chemotherapies or biological therapies, namely standard or experimental chemotherapies, or immunotherapies or ionising radiations in admixture or combination with one or more non-toxic, inert, pharmaceutically-acceptable diluent(s) or carrier(s).

The present invention also relates to pharmaceutical compositions wherein the immunologically-active ingredient is a triacylated diphosphorylated lipid A derivatives of structural formula (III):

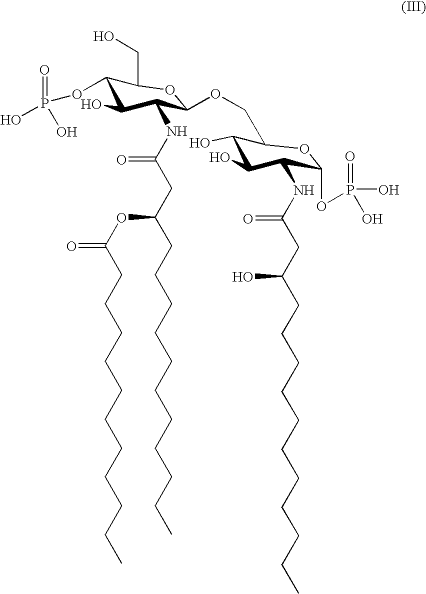

in conjunction or admixture with an inert, non toxic pharmaceutically-acceptable carrier or vehicle.

This invention also relates to methods for treating cancer in warm blooded animals including humans suffering from cancer, which consists in administering to them a combination of a therapeutically effective amount of a mixture of compounds of general formula (I)

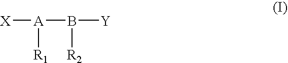

wherein
X, Y, A, B, R₁ and R₂ have the above-given definitions,
in combination with a known antineoplastic agent selected from the group consisting either of:
1) accepted or experimental radiation therapy techniques, or radiations source or chemosensitizer
2) and one or more agent(s) selected from the group consisting of, as a chemotherapeutic agent, an alkylating agent, an antimetabolite agent, an agent acting on tubules, a tyrosine-kinase inhibitors, in a pharmaceutically-acceptable carrier excipient or vehicle suitable for the oral, parenteral, rectal, topical, subcutaneous or sub-mucosal ways.

The active ingredients may be given either simultaneously mainly in a single unit dosage, or separately or sequentially in separate unit dosages, mainly as a kit containing in separate containers the various active ingredients.

These pharmaceutical compositions and the method using the same are based on well established agents as well as newly developed methods to treat neoplastic diseases.

### PRIOR ART

### Control of Cancer by the Immune System

Healthy cells normally divide, grow, and finally die when necessary in a patterned and well controlled manner. Often during a life-time it happens incidentally that an individual cell starts to divide without control. Since nature is well prepared, the generated uncontrolled cells concomitantly generally express on their surface modified antigens (tumor associated antigens) which are normally not present on non-tumor cells, allowing thus in the vast majority of the cases, the immune system to prevent the apparition of many cancers.

### Cancer Cells May Escape Immune Recognition

However, some cancer antigens are tissue-specific molecules shared by cancer cells and healthy cells. Thus, these weak antigens do not typically elicit immunity. In addition, tumors have several features that make their recognition and destruction by the immune system difficult. Indeed cancer cells are known to release immunosuppressive substances (such as e.g. the cytokine TGF-beta or the prostaglandin PGE₂ to escape immune recognition.

If the immune system, for any reason, fails to recognize the danger and to destroy the proliferating cells, cancer and metastases appear.

Combining Immunotherapy with Standard Chemotherapeutic Drugs

When cancer is established, it is unfortunately often incompletely treated by rather aggressive chemotherapeutic drugs or radiotherapeutic methods which may further damage the already weakened human immune system.

The general practice today is to use immunostimulation (e.g by filgrastim or NEUPOGEN®, a medication that stimulates blood cell proliferation to fight the potential complications of neutropenia), principally to restore the immune system often severely damaged by the chemotherapeutic agent used, or after radiotherapy. The common standard rational is to use immunostimulating agents in order to restore “normal” blood cellular formulas to avoid as much as possible opportunistic infections in cancer patients undergoing an anticancer therapy.

In contrast, in this application it is proposed that a clinical treatment with a triacylated lipid-A derived immunostimulating agent, takes place before, concomitantly, or after the use of well-established standard or experimental anticancer cytotoxic drugs or radiotherapeutic treatments in order to improve the efficacy of the anticancer treatment as shown in the examples below.

### The Burden of Cancer

Cancer presently refers to a family of related proliferative diseases, which kill millions of persons each year. Despite recent progresses such as the use of Gleevec®, effective therapeutic agents to fight cancer, continue to be lacking, and cancer rates could further increase by 50% to 15 million new cases in the year 2020, (World Cancer Report, www.who.int/mediacentre/releases/2003/pr27/en/-40k).

In the year 2000, malignant tumours were responsible for 12 percent of the nearly 56 million deaths worldwide from all causes. In 2000, 5.3 million men and 4.7 million women developed a malignant tumour and altogether 6.2 million died from the disease. Cancer remains the third letal cause, after infectious and parasitic diseases on one part and coronary and heart diseases on the other part.

Lung cancer is the most common cancer worldwide, accounting for 1.2 million new cases annually; followed by cancer of the breast, just over 1 million cases; colorectal, 940,000; stomach, 870,000; liver, 560,000; cervical, 470,000; esophageal, 410,000; head and neck, 390,000; bladder, 330,000; malignant non-Hodgkin lymphomas, 290,000; leukemia, 250,000; prostate and testicular, 250,000; pancreatic, 216,000; ovarian, 190,000; kidney, 190,000; endometrial, 188,000; nervous system, 175,000; melanoma, 133,000; thyroid, 123,000; pharynx, 65,000; and Hodgkin disease, 62,000 cases.

The three leading causes of cancer are different than the three most common forms, with lung cancer responsible for 17.8 percent of all cancer deaths, stomach, 10.4 percent and liver, 8.8 percent.

### Main Treatments to Combat Cancer

Most cancers are classically treated with
surgery,
radiation therapy,
chemotherapy,
and/or biological therapy.

### Surgery:

During this procedure, solid tumoral masses are removed from the body. However if metastases have already spread out, this treatment procedure becomes usually useless.

### Radiation Therapy

This method, also called radiotherapy, refers to the use of high-energy radiation from X-rays, gamma rays, neutrons, and other sources to kill cancer cells and shrink tumors.

It may be given before surgery (neoadjuvant therapy) to shrink a tumor so that it is easier to remove. In other cases, radiation therapy is given after surgery (adjuvant therapy) to destroy any cancer cells that may remain in the area.

Interestingly, it has been recently demonstrated (De Ridder et al., Int J Radiat Oncol Biol Phys. 2003 Nov. 1; 57(3):779-86) that hypoxic breast tumour cells (EMT-6 cells) display increased radiosensitivity (from 0 to 20 Gy) 16 h after NF-kB (and therefore nitric oxide) activation. As triacylated lipid-A derivatives have been shown to induce even higher nitric oxide levels from macrophages than LPS, it is claimed here that triacylated lipid-A derivatives would be potent anticancer agents when used in combination with radiotherapy. Macrophages enhance the radiosensitizing activity of lipid A (de Ridder et al., Int J Radiat Oncol Biol Phys. 2004 Oct. 1; 60(2):598-606), thus suggesting a novel role for immune cells in tumor cell radioresponse. The effect of one triacylated lipid-A derivative according to the general formula I is presented below in such a system.

### Chemotherapy:

Chemotherapy is usually given in cycles: a treatment period, one or more days, followed by a recovery period, several days or weeks, then another treatment period, and so on. Here, it is proposed that in between or concomitantly to these chemotherapeutic cycles (designed to shrink the tumour and reveal tumour antigens), the stimulation of the immune system by triacylated compounds of the invention could be performed.

### The Rational Behind Chemotherapy:

Any efficient and safe chemotherapy drug should kill the cancer cells and not harm the adjacent healthy cells. This can in theory be achieved by characterizing properties unique to cancer cells which are not found on normal tissues.

The strategy behind the clinical use of chemotherapeutic drugs, is based on the simple factual observation that most cancer cells grow faster than normal cells. Therefore targeting specifically some enzymes or cellular elements involved in the cell growth cycle, seems reasonable. This cytotoxic strategy implies that fast growing cells would be most affected, and slow growing cells would be less disturbed. This rational was indeed applied for the development of many chemotherapeutics currently used clinically.

Chemotherapeutic agents are mainly active during the S and M phases of the cell cycle.

### The Limits of Chemotherapy:

Beside it's still largely insufficient clinical efficacy, this strategy has its own toxicological limitations, because some normal cells (such as e.g. proliferating T and B cells) need also to divide when necessary. Indeed, when a patient suffers from kidney or liver damage and can therefore not eliminate normally a chemotherapeutic agent, administering the recommended amount of drug may prove to be too toxic in a patient unable to metabolize and/or excrete it. Therefore dose adjustments are an absolute necessity to avoid non-acceptable toxicities or sub-therapeutic dosing.

The pharmacokinetics for cancer patients are often very complex, and sometime limits the patient's chemotherapy options.

### How to Enhance the Efficacy of Chemotherapy and Reduce Side-Effects:

It is contemplated here that an adequate and timely controlled clinical combined therapy with well-recognized or experimental chemotherapeutic drugs, used first to shrink and kill some cancer cells (and thus potentially reveal tumour-associated antigens), followed by an unspecific immunostimulation with a triacylated compound of the present invention enhances the efficacy of the oncostatic drug, and permits the acquisition of an immunological (specific) memory to get rid of cells bearing the tumour associated antigen, and also to limit the level of the sideeffects observed, by allowing e.g. to reduce the number of administrations and/or the doses of the chemotherapeutic drug.

### Major Chemotherapeutic Drugs:

Text adapted from

A Chemotherapy Primer: Why? What? and How?, Julia Draznin Maltzman, M. D, Nov. 5, 2003, OncoLink, Abramson Cancer Center of the University of Pennsylvania

Chemotherapeutic agents can be divided into the following classes:
Alkylating agents:
   For example, Alretamine, BCNU, Busulfan, Carmustin, CCNU, Chlorambucil, Chlormethin, Carboplatin, Cisplatin, Cyclophosphamide, Dacarbazine, Estramustin, Fotemustin, Ifosphamide, Lomustin, Maphosphamide, Melphalan, Mitomycin, Nimustin, Oxaliplatin, Procarbazine, Streptozocin, Thiotepa, Lobaplatin, Miboplatin, and so on.
Intercalants/topoisomerase II inhibitors
   Asacrin, Dactinomycin, Daunorubicin, Doxorubicin, Elliptinium Acetate, Epirubicin, Idarubicin, Mitoxanthrone, Pirarubicin.
   Plicamycine
   Vabrubicine
   Zorubicine
   and so on.
   They are known for possessing a high and irreversible cardio-toxicity.
Topoisomerase I inhibitors
   Irinothecan and Topothecan
Antimetabolites
   They are subclassified into three classes
antifolic agents,
purine analogs,
and pyrimidine analogs.

Examples thereof are Capecitabine, Cladribine, Cytarabine, Fludarabine, Fluorouracil (5-FU), Gemcitabine, Mercaptopurine, Methotrexate, Thioguanin and the like.
Agents acting on tubules: (e.g alcaloids and toxoids)
   Paclitaxel, Docetaxel, Taxol, Vinblastine, Vincristine, Vindesine, Vinorelbine and the like . . . .
Tyrosine kinase inhibitors:
   Protein kinase inhibitors are used as anticancer therapeutic agents and biological tools in cell signaling. Two representative members of this family of compounds are Imatinib Mesylate (Gleevec®3) and Gefitinib (Iressa®).
Other chemotherapeutic agents: They are enzyme or antibiotics such as:
Asparaginase,
Bleomycin,

### Alkylating Agents:

Alkylating agents share a common mechanism of action to the poisonous nitrogen mustards compounds originally developed for military use. It is therefore not surprising that such agents display a full array of adverse events.

They act on the negatively charged sites on DNA. By linking to DNA, replication and transcription are altered, cellular activity is stopped, and cells start to die. This class of anticancer drugs is very powerful and is used in many types of cancer (both solid tumors and leukemia). Unfortunately, the side effects noted are considerable (mainly decreased sperm production, cessation of menstruation, and possibly cause permanent infertility). Alkylating agents can cause secondary cancers. The most common secondary cancer is a leukemia (Acute Myeloid Leukemia) that may occur years after the end of the therapy.

Natural metal derivatives such as the platinum derivatives, for example cisplatin have demonstrated some activity against cancer, mainly against lung and testicular cancer. The most significant toxicity of cisplatin is kidney damage. Second-generation platinum derivatives, called carboplatin, have fewer kidney sideeffects, and may be an appropriate substitute for regimens containing cisplatin. Oxaliplatin is a third-generation platinum that is active in colon cancer and has no renal toxicity. However, its major sideeffects are neuropathies.

It is provided below, examples in different models, in which the use of a triacylated lipid-A analog after treatment with alkylating agents such as cyclophosphamide or cisplatin display a very good synergistic antitumoral activity. In the “in vivo” examples provided (see appropriate sections), in the conditions used, each agent individually does not give satisfactory anticancer results, and quite unexpectedly, a non specific boost of the immune system by triacylated lipid-A derivatives after a first non specific chemotherapeutic treatment provides encouraging anticancer results worth to be tested in clinical anticancer trials.

### Intercalants/Topoisomerase II Inhibitors:

These compounds form a complex with the enzyme and the DNA, and therefore inhibit DNA re-ligation. They are used to treat mainly malignant hemopathies, breast cancer, digestive tract cancers, genital cancers, bronchial, or conjunctive sarcomas. Their main adverse events are myelo-suppression, vomiting, cardiotoxicity, and alopecia.

### Topoisomerase I Inhibitors:

They inhibit specifically topoisomerase-I, and thus transcription and replication during the S-phase of the cell-cycle.

They are mainly used to fight colorectal cancers. Their main adverse events are myelo-suppression, neutropenia, vomiting, alopecia, and cholinergic syndromes.

### Antimetabolites:

They are used mainly against trophoblastic carcinomas, breast cancer, ovarian cancer, acute leukemia, osteosarcomas, lymphomas . . . .

Their main adverse events concern mainly myelosuppression, mucites, cutaneous toxicity, diarrhea, vomiting . . .

In 1948 Farberdemonstrated that a folic acid analog could induce remission in childhood leukemia. Then other analogs inhibiting key enzymatic reactions were synthetized. Antimetabolites interfere with normal metabolic pathways, including those necessary for making new DNA (phase S of the cell cycle). The most widely used antifolate in cancer therapy with activity against leukemia, lymphoma, breast cancer, head and neck cancer, sarcomas, colon cancer, bladder cancer and choriocarcinomas is Methotrexate which inhibits a crucial enzyme (dihydrofolate reductase) required for DNA synthesis.

Another widely used antimetabolite that disturbs DNA synthesis is the pyrimidin analogue 5-Fluorouracil, which is transformed in fluorodeoxiuridin monophosphate (5-FdUMP) which blocks the enzyme thymidilate synthase, necessary for the endogenous synthesis of pyrimidin bases (C and T). An example of combination of a triacylated compound according to the general formula I with 5-Fluorouracil to treat colon cancer will be provided below. The compound has a wide range of activity including colon cancer, breast cancer, head and neck cancer, pancreatic cancer, gastric cancer, anal cancer, esophageal cancer and hepatomas. However, 5-Fluorouracil is metabolized by the enzyme dihydropyrimidine dehydrogenase (DPD), which is not expressed by a small population of patients. When these patients are challenged with this chemotherapeutic drug, they get acute and severe toxicity (bone marrow suppression, severe GI toxicities, and neurotoxicities which may include seizures and even coma). Capecitabine is an oral pro-5-Fluorouracil compound that has similar side-effect potentials. Premetrexed is an antifolate antineoplastic agent impeding cell replication intended for injection (Alimta®), produced by Eli Lilly and Company.

Other antimetabolites that inhibit DNA synthesis and DNA repair include: Cytarabine, Gemcitabine (Gemzar®), 6-mercaptopurine, 6-thioguanine, Fludarabine, and Cladribine.

### Agents Acting on Tubules: (e.g Alcaloids and Toxoids)

Alcaloids such as Vinblastine, Vincristine, Vindesine, or Vinorelbine bind to tubulin, a cytoplasmic protein and therefore impede the formation of the mitotic spindle and block mitosis in the metaphase.

Vincristine, vinblastine, and vinorelbine were extracted from the leaves of a periwinkle plant, Vinca rosea. They are mainly used to treat malignant hemopathies (including Hodgkin), aero-digestive cancers, nephroblastomas, breast cancers . . . .

Their main adverse effects are myelosuppression, nausea, vomiting, alopecia, causticity, neuropathy and neurotoxicity.

Taxanes, first isolated from the bark of the Pacific yew tree Taxus brevifolia in 1963, are specific for the M phase of the cell cycle. The family includes paclitaxel and docetaxel. Taxanes bind with high affinity to the microtubules and inhibit their normal function. They are efficient against breast cancer, lung cancer, head and neck cancer, ovarian cancer, bladder cancer, esophageal cancer, gastric cancer and prostate cancer. These drugs however lower the number of blood cells.

Their main adverse effects are mainly myelosuppression (neutropenia), and lymphoedema

### Tyrosine Kinase Inhibitors

The Tyrosine kinase inhibitor Gefitinib (Iressa®, AstraZeneca) is used for treatment of advanced non-small cell lung cancer (NSCLC), the most common form of lung cancer in the United States.

Gefitinib blocks the action of the EGF receptors on the cells of certain lung cancers and has shown some effects against these cancers.

Some common side effects with Iressar® include among others: diarrhea, rash, acne, dry skin, nausea, vomiting, itching, loss of appetite, weakness, and weight loss.

The tyrosine kinase inhibitor Imatinib Mesylate (Gleevec®, Novartis) has been approved for the treatment of patients with positive inoperable and/or metastatic malignant gastrointestinal stromal tumors (GISTs) and for the treatment of chronic myeloid leukemia (CML).

Imatinib Mesylate is a signal transduction inhibitor that acts by targeting the activity of tyrosine kinases. The activity of one of these tyrosine kinases, known as c-kit, is thought to drive the growth and division of most GISTs. Imatinib is an inhibitor of the receptor tyrosine kinases for platelet-derived growth factor (PDGF) and stem cell factor (SCF), c-kit, and inhibits PDGF- and SCF-mediated cellular events. In vitro, imatinib inhibits proliferation and induces apoptosis in GIST cells, which express an activating c-kit mutation.

The majority of patients who received Gleevec® in clinical studies did experience sideeffects, such as nausea, fluid retention (swelling around the eyes, of the legs, etc.), muscle cramps, diarrhea, vomiting, hemorrhage, muscle and bone pain, skin rash, headache, fatigue, joint pain, indigestion, and shortness of breath.

### Other Chemotherapeutic Agents:

Bleomycin is a small peptide isolated form the fungus Streptomyces verticillus. Its mechanism of action is similar to that of anthracyclines. Free oxygen radicals are formed that result in DNA breaks leading to cancer cell death. This drug is rarely used by itself rather in conjunction to other chemotherapies. Bleomycin is an active agent in the regimen for testicular cancer as well as Hodgkin's lymphoma. The most frequent side effect of this drug is lung toxicities due to oxygen free radical formation.

Asparaginase catalyses the hydrolysis of asparagin in aspartic acid and ammonium, and therefore can kill cancer cells sensitive to a lack of asparagine-synthetase (lymphocytes and cells of lymphoid origin). It is used to treat hemopathies (acute leukemias, non Hodgkin lymphomas . . . ). Its main adverse events are hepatic toxicity, nausea, and some anaphylactic shocks.

### Biological Therapy:

This section has been divided in 3 parts: Monoclonal antibodies, cytokines, and immunostimulation by bacterial agents. The compounds of this invention belong to this class of agents.

### Monoclonal Antibodies:

Mouse, chimeric, humanized and human monoclonal antibodies (huMoAb) are used for treatment of human cancer [Untch M, Ditsch N, Hermelink K., Immunotherapy: new options in breast cancer treatment. Expert Rev Anticancer Ther. 2003 June; 3(3):403-8].

It is estimated that about 20 antibodies will be in clinical use by the year 2010.

The use of monoclonal antibodies involves the development of specific antibodies directed against antigens located on the surface of tumor cells. Samples of the patient's tumor cells are taken and processed to produce specific antibodies to the tumor-associated antigens. In order for this approach to work, a sufficient quantity of antigens unique to the tumor cells must be present. In addition, the tumor antigens must be sufficiently different from the antigens elaborated to by normal cells to provoke an antibody response.

These antibodies (recognizing cancer cells) can be used either alone to kill cancer cells or as carriers of other substances used for either therapeutic or diagnostic purposes. For example, chemotherapeutic agents can be attached to monoclonal antibodies to deliver high concentrations of these toxic substances directly to the tumor cells. In theory, this approach is less toxic and more effective than conventional chemotherapy because it reduces the delivery of harmful agents to normal tissues.

Erbitux (cetuximab) is a monoclonal antibody that targets epidermal growth factor receptor (EGFR), and thus regulates cell growth. Erbitux is believed to interfere with the growth of cancer cells by binding to EGFR so that endogeneous epidermal growth factors cannot bind and stimulate the cells to grow. Erbitux is used to treat metastatic colon or rectum cancers. The infusion of Erbitux can cause serious side-effects, which may include difficulty in breathing and low blood pressure, which are usually detected during the first treatment. Infrequent interstitial lung disease (ILD) has also been reported. Other more common side effects of Erbitux treatment are: rash (acne, rash, dry skin), tiredness/weakness, fever, constipation, and abdominal pain.

Rituximab (anti-CD20) was the first registered MAB for the therapy of follicular lymphoma. Impressive results have been seen in combination with CHOP chemotherapy (cyclophosphamide, doxorubicin, vincristine and prednisone) in follicular and high-grade lymphomas.

Other marketed monoclonal antibodies are: Alemtuzamab (Campath®, targets CDw52 expressed on lymphoid tumors); Gemtuzumab-ozogamicin (Mylotarg® targets CD33 expressed on myeloid leukemia blasts), and Tositumab (Bexxar®).

### Cytokines:

The main cytokines tested for the treatment of cancer are Interleukin-2 and interferons.

### Interleukin-2:

Interleukin-2 (IL-2) is a substance produced by lymphocytes. In addition to being an essential growth factor for T cells, IL-2 increases various NK and T-cell functions. IL-2 also activates lymphokine-activated killer (LAK). LAK cells destroy tumor cells and improve the recovery of immune function in certain immunodeficiency states. Patients with renal cell cancer, melanoma, and non-Hodgkin's lymphoma have demonstrated some responses to IL-2 therapy.

The most severe toxicities result from IL-2's ability to increase capillary permeability. This may cause hypotension, ascites, generalized body edema, and pulmonary edema. Chills and fever also frequently occur within a few hours after IL-2 administration. Headache, malaise, and other flu-like symptoms are also common. Gastrointestinal effects include nausea, vomiting, loss of appetite, diarrhea, and mucositis.

### Interferons:

Interferons (IFNs) are small proteins that inhibit viral replication and promote the cellular (T-cell) immune response. There are currently three major types of IFNs: alpha, beta, and gamma. Each type has similar but distinctive capabilities for altering biological responses. Alpha-IFN main indication is for use in treatment of hepatitis C, but it is currently also indicated for use in the treatment of hairy cell leukemia and AIDS-associated Kaposi's sarcoma. It also displays some therapeutic effectiveness against hematologic diseases such as low-grade Hodgkin's lymphoma, cutaneous T-cell lymphoma, chronic myelogenous leukemia, and multiple myeloma. It is also somewhat effective on some solid tumors, such as renal cell cancer.

Beta-interferon is currently in use for treatment of multiple sclerosis.

One of the most common side effects of IFN therapy is a flu-like syndrome. Symptoms include fever, chills, tachycardia, muscle aches, malaise, fatigue, and headaches.

Other common side effects to IFN include a decrease of the white blood cell count, anemia (with prolonged therapy), and decreased platelets. Gastrointestinal symptoms such as a loss of appetite, nausea, vomiting, and diarrhea may also be present. Central nervous system toxicities range from mild confusion and sleepiness to seizures. Acute kidney failure is rare, but can occur. Loss of hair may also be a problem.

### Immunostimulation by Bacterial Agents:

After promising results in animal studies during the sixties, searchers initiated large-scale clinical trials to stimulate cancer patients' immune systems using bacterial agents such as Corynebacterium parvum (C. parvum) and Bacillus Calmette-Guerin (BCG). Unfortunately, the results of these early immunotherapy trials were discouraging, and cancer treatment using immunostimulating drugs per se lost momentum.

The toxicity of extrinsic immuno-stimulants strongly limited their use in cancer patients. In 1976, Morales et al introduced intravesical Bacillus Calmette-Guérin (BCG) to treat superficial bladder cancer (Morales et al. 1976, rediscussed in J Urol. 2002 February; 167(2 Pt 2):891-3; discussion 893-5.). BCG, a non-specific immunotherapy for superficial bladder cancer may be regarded as the most successful of all immunotherapies in man (for recent review see Boyd, Urol Nurs. 2003 June; 23(3):189-91, 199; quiz 192.).

The antitumour effect of lipopolysaccharides (LPS) has been well established. In the 19th century Coley developed a cancer therapy based on bacterial toxins (see Coley W B, the Practitioner, November 1909). In the 1940's it was shown that bacterial lipopolysaccharide (LPS) was at least partially responsible for the observed anti-tumour activity in Coley's toxins. More recent publications have shown anti-tumoural effects of LPS in animal models and a very limited number of studies have been carried out in man. Because LPS is very toxic and can lead to endotoxic shock, the therapeutic window appears to be very small, and patients can only be treated using very small amounts of LPS that are often too low to obtain the desired beneficial effects.

The biological and toxic activities of LPS are associated with its lipid moiety, called lipid A. Different bacterial species synthesize different lipid A structures and these have varying degrees of toxicity. This suggests that by modifying the structure of the native bacterial lipid A, it would be possible to prepare derivatives that have attenuated toxicity but retain beneficial biological activity. A number of different lipid A derivatives have been tested in animal models of cancer with some success.

Presently it is proved that immunostimulation with OM-174 a triacylated diphosphorylated lipid A derivatives of structural formula (III) would help the body's immune system to achieve a coordinated combination of nonspecific and specific responses to tumor associated antigen if these are revealed first or concomitantly by a classical chemotherapeutic agent as those described above.

Once the first chemotherapeutic treatment has been performed, it would be necessary to initiate an inflammatory response to boost first the nonspecific host defense. Then, specific immune responses would be elicited by the presence of the revealed tumour associated antigen. These specific memory responses are generally divided into humoral (immunity conferred by the antibodies produced by B-lymphocytes) and cell-mediated immunity (immunity conferred by T-lymphocytes). Other important cells are antigen presenting cells (APC) such as macrophages and natural killer (NK) cells. Macrophages bind to an antigen and “present” the antigen to naive T-cells. These, in turn, become activated and produce mature lymphocytes. NK cells are cytotoxic to tumor cells and virus-infected cells.

Contemplated Combined Treatments with Triacylated Lipid-A Derivatives:

The goal of the present therapeutic strategy to fight cancer is to first attack cancer cells with standard or experimental chemotherapeutic drugs, and thus reveal “in situ” cancer antigens, and to subsequently boost the immune system to prepare an appropriate immunological response. Alternatively, radiotherapy rather than chemotherapy could be also used. Moreover the synergistic use of an immunostimulating cytokine (such as alpha-IFN) and a triacylated lipid-A derivative could be envisaged to boost ex-vivo or in vivo the maturation and activation of human monocyte-derived dendritic cells as described by B. Veran J., M. Mohty B. Gaugler, C. Chiavaroli and D. Olive. 2004, Immunobiology 209:67.

The aim of the invention when compared to the “current art” resides in the fact that, according to applicant's knowledge, no animals experimental studies have been disclosed on the effects of combining any triacylated diphosphorylated lipid A derivatives of structural formula (II) with any standard chemotherapeutic drug claimed here, and the use of the two nonspecific agents, as a standard or experimental chemotherapeutic drug, and the immunostimulating agent, lead to an efficient specific (antigens revealed by the chemotherapy) anticancer treatment.

The present invention resides in the fact that triacylated lipid-A derivatives could be used therapeutically to treat many forms of cancer in combination with the compounds and drugs listed below, or in combination with radiotherapy.

### The Triacylated Lipid-A Derivative OM-174 in Man

The product was well tolerated in cancer patients. Doses higher than 1 mg OM-174/m² by i.v. infusion were reached without unacceptable toxicity according to non-haematological grade III and haematological grade IV NCI Common Toxicity Criteria.

The analyzed cytokines (TNF-α, IL-1b, IL-1 ra, IL-6, IL-8, sTNF-RI, sTNF-RII, IL-10, IL-2, IL-2sRa, IFN-γ) showed a secretion profile consistent with that of lipid A derivatives. Secretion occurred in all steps, and appeared more “patient”- than “dose”-dependent.

The results of this single dose study led to the selection of three doses (0.6, 0.8, and 1.0 mg OM-174/m²) for repeated i.v. injections (5 to 15 injections), used in a phase Ib study.

Pharmacokinetic data in man (clearance, volume of distribution, and half-live) are summarized in the Table 1 for OM-174

TABLE 1
Summary of pharmacokinetic data of OM-174 in man

| | Healthy volunteers | Cancer patients |
|---|---|---|
| | (median and range) | (median and range) |
| CL (ml/hr) | 169 (116-202) | 102 (55-173) |
| Vₛₛ (l) | 5.1 (4.3-6.8) | 2.9 (1.9-4.8) |
| T_{1/2} (hr) | 23 (18-32) | 20 (12-28) |

List of Drugs Likely to be Combined with the Compounds of the Invention:

Alemtuzamab; Alretamine; Asacrin; Asparaginase (Elspar®); Anastrozole (Arimidex®), Bevacizumab (Avastin®); Bicalutamide (Casodex®); Bleomycin (Blenoxane®); Bortezomib (Velcade®); Busulfan (Myleran); Capecitabine (Xeloda®); Carboplatin (Paraplatin); Carmustine (BCNU, BiCNU); Cetuximab (Erbitux®); Chlorambucil (Leukeran); Chlormethin; Cisplatin (Platinol®); Cladribin; Cyclophosphamide (Cytoxan®, Neosar®); Cytarabine (Cytosar-U®, Ara-C); Dacarbazine (DTIC-Dome); Dactinomycin (Cosmegen®); Daunorubicin (Cerubidine®); Dexrazoxane (Zinecard®); Docetaxel (Taxotere®); Doxorubicin (Adriamycin, Rubex); Erbitux (cetuximab), Elliptinium acetate; Epirubicin; Estramustin; Etoposide (VePesid®, VP-16®); Fentanyl Citrate (Actiq); Floxuridine (FUDR®, Fluorodeoxyuridine); Fotemustin; Fludarabine (Fludara®); Fluorouracil (Adrucil, 5-FU); Flutamide (Eulexin®); Fulvestrant (Faslodex®); Gefitinib (Iressa®) Gemcitabine (Gemzar®); Gemtuzumab; Goserelin acetate implant (Zoladex®); Hydroxyurea (Hydrea®); Idarubicin (Hydrea®); Ifosfamide (IFEX®); Imatinib Mesylate (Gleevec, STI-571); Irinotecan (Camptosar®, CPT-11); Leucovorin; Leuprolide acetate for depot suspension (Lupron®); Lomustine (CCNU, CeeNU®); Maphosphamide; Mechlorethamine (Mustargen®, Nitrogen Mustard); Melphalan (Alkeran®, L-PAM); Mercaptopurine (Purinethol®, 6-MP); Methotrexate (MTX); Mitomycin (Mitomycin C, Mutamycin); Mitotane (Sodren); Mitoxantrone (Novantrone); Nilutamide (Nilandron®); Oxaliplatin (Eloxin®); Paclitaxel (Taxol); Pamidronate (Aredia); Pentostatin (Nipent); Pirarubicin; Plicamycin (Mithracin, Mithramycin); Premexetred (Alimta®); Procarbazine (Mutalane); PROCRIT (Epoetin alfa); Polifeprosan 20 with carmustine implant (GLIADEL®); Rituximab (Rituxan®); Streptozocin (Zanosar); Tamoxifen (Nolvadex®); Teniposide (Vumon); Tepotecan; Thioguanine (6-TG, Thioguanine Tabloid®) Thiotepa (Thioplex); Tositumomab (Bexxar®); Toxaliplatin (Elotaxin®); Vinblastine (Velban); Vincristine (Oncovin); Vindesine; Vinorelbine (Navelbine)

### DESCRIPTION OF THE INVENTION

The compounds of the invention are obtained according to the process described in WO 95/14026.

The compounds of the invention can be in the form either of the acid form or of any acceptable salt suitable for injection in warm blooded animals and human beings. Compounds will be administered parenterally (i.v. preferentially) after (or concomitantly in any suitable formulation) a preliminary therapy involving standard radiotherapy or classical or experimental chemotherapeutic drugs.

In humans first, tumours would be treated conventionally with well defined or experimental chemotherapeutic agents or radiotherapy to reveal the patients tumour antigens. Then (or concomitantly) immunostimulation with the compounds of the invention (preferentially 1 to 7 injections/per week and at least 5 parenteral injections) will be performed. Cycle of conventional therapies could then be performed optionally with decreased doses.

It has been known from previous work as disclosed in WO 95/14026 that when tested per se as an immunotherapeutic agent, OM-174 displays a strong therapeutic activity even when treatment, in the BDIX/ProB colon model of cancer, is started up to 14 days after tumour induction. Such a treatment leads either to cure or to give strong inhibition of tumour development. In the case of complete remission, animals are immunized specifically against the tumour, and re-implantation leads to rejection. Treatment consisted of repeated injections of OM-174, the schedule of administration being more critical than the dose for the therapeutic effect of the drug.

It will be shown below that there is potentially a major advantage in combining the effects of immunotherapy (induced e.g. by OM®-174) with those of chemotherapy or radiotherapy. Thus, an initial treatment for cancer by—for example—chemotherapy (alkylating agents such as cisplatin analogues or cyclophosphamide, or antimetabolite agents such as 5-FU), will reduce the tumour mass and viability, and by damaging the tumour cells, may also render them more immunogenic. This initial non specific treatment could then be followed by non-specific immunotherapy by the compounds of the invention, which would be more effective as a result of the initial chemotherapy. Immunotherapy will lead to the specific rejection of remaining tumour cells by the immune system, the prevention of any tumour regrowth and metastatic growth.

This combination of treatments potentially offers a very powerful method to fight cancer as described in the examples below.

The impact of such an invention is broad, when one considers the number of anticancer agents and cancer types. The clinical model for phase II studies will involve administration of OM-174 or other triacyl derivatives (bolus+infusion) concomitantly or after chemotherapeutic agents or radiotherapy.

Advantages and improvement due to the specified therapy will more clearly appear from the examples attached herewith and the appended claims.

### EXAMPLES

### Example 1

### Enhancement of the Curative Effect of Cyclophosphamide by OM-174 in the Melanoma B16 Model

### Introduction

To present knowledge, no experimental studies have been disclosed on the effects of combining OM-174, a triacylated diphosphorylated lipid A derivative of structural formula (II) with standard chemotherapeutic drugs as those claimed in this document.

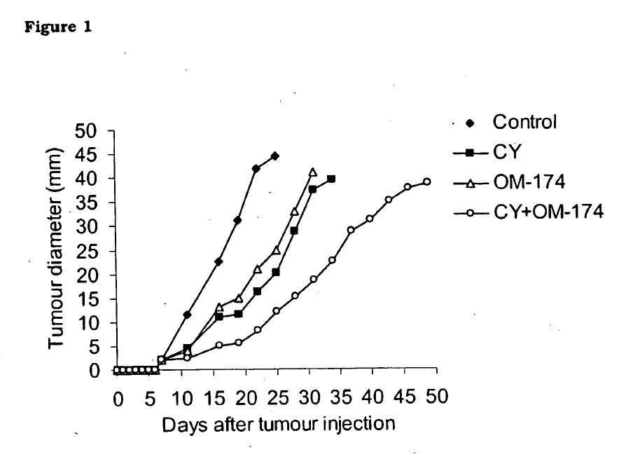

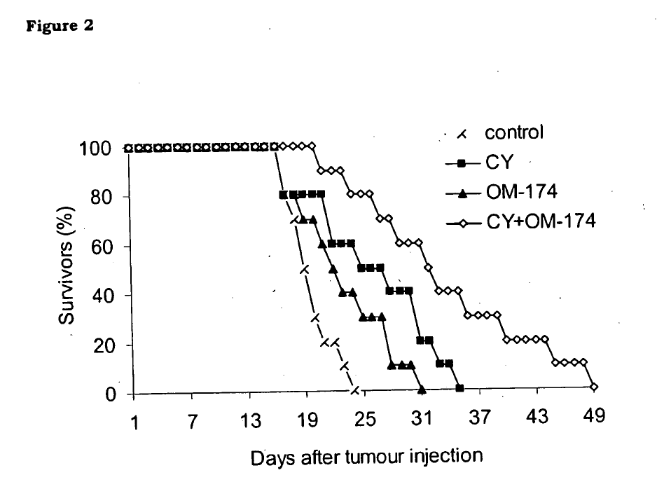

In this example, it is shown that OM-174 per se partially inhibits tumour progression (FIG. 1) and slightly extends the survival time of mice in the B16 melanoma experimental model (FIG. 2). In the conditions used in the study, OM-174 antitumour activity is comparable to that of cyclophosphamide (CY), a reference cytostatic drug.

Interestingly, and this is a part of the invention, more striking effects are achieved by means of the combination of the two agents in a protocol consisting of a single administration of CY (200 mg/Kg, i.p.) followed by five injections of OM-174 (1 mg/Kg, i.p.). See FIGS. 1 and 2.

Immunological studies of treated and control mice revealed that the antitumour activity of OM-174, alone or in combination with CY, is mediated by the stimulation of natural killer (NK) and cytotoxic T lymphocyte (CTL) responses as well as by a significant increase in the absolute number of NK1.1, CD4 and CD8 positive cells. OM-174 therefore increases the anticancer effect of the well-known chemotherapeutic drug cyclophosphamide and is therefore a candidate for association with chemotherapy in the treatment of human cancers.

### Animals and Tumour Cells

Four to six weeks-old male C57BL/6 mice were purchased from Charles River (Calco, Corno, Italy). B 16 melanoma tumour cells were serially passaged subcutaneously (s.c.) in syngenic mice. On day 0, mice were injected s.c. in the right flank with 2×10⁵ B16 melanoma cells. Tumour growth was measured daily in each mouse, using calipers, and mean tumour diameter per day was calculated. At day 7 after tumour injection, all mice with s.c. tumours of about 2-3 mm diameter were divided into different experimental groups, i.e. phosphate buffered saline (PBS)-injected control 3, CY, OM-174 or CY with OM-174.

### Drugs and Treatments

Cyclophosphamide (Sigma, St. Louis, Mo.) was dissolved at 20 mg/ml in PBS immediately before use, and 0.2 ml per mouse were injected intraperitoneally. Each treated animal received a single dose of 200 mg/Kg CY on day 7. This dose was chosen on the basis of previous experiments as the most active one, that did not lead to observable toxicity in this strain of mice.

Immunostimulating agent OM-174, is a purified water soluble diphosphorylated and triacylated lipid A derived from E. coli. For the study of tumour growth and survival, each mouse (20/group) received OM-174 i.p. (1 mg/kg) on days 8, 13, 18, 23 and 28 after tumour inoculation. The analysis of the spleen cell cytotoxic activities and lymphocyte subsets of different experimental groups (5 animals/group) was performed on day 14 after tumor injection, i.e. after two treatments with OM-174 (on days 8 and 13).

### Spleen Cell Preparation:

Mice were sacrificed by cervical dislocation on day 14 after tumour inoculation. Spleen cells were obtained by gently teasing the individual spleens in RPMI 1640 (Flow Laboratories, Irvine, Ayrshire, U.K.). Cells were filtered through a 10 μm Nytex mesh, then washed twice and resuspended in Complete Medium (CM): RPMI 1640 supplemented with 10% foetal bovine serum (FBS), 200 mM L-glutamine, 25 mM HEPES, penicillin 50 U/ml and streptomycin 50 μml (all from Flow Laboratories).

### Cytotoxicity Assays:

In vitro-passaged YAC-1 cells (a Moloney-virus induced mouse T cells lymphoma of A/SN origin), and in vivo-passaged B16 melanoma cells, were used as target cells in a chromium-release assay. B16 melanoma cells were obtained from tumour-bearing mice, seeded in cell-culture flasks (Falcon, Becton Dickinson and Co., Plymouth, England) and used within the first week of culture in CM. B16 and YAC-1 cell lines were obtained from the laboratory collection, and were originally obtained from the American Tissue Culture Collection (ATCC).

The cytotoxic activity of the effector cells collected from individual mice was measured by a standard 4-hour ⁵¹Cr-release assay. Briefly, target cells were harvested from the cultures, washed twice, resuspended at 5×10⁶ cells in 0.9 ml of CM and labelled with 100 μCi (⁵¹Cr) sodium chromate (New England Nuclear, Boston, Mass.) for 1 hour at 37° C. in a 5% CO₂ incubator. After labelling, the cells were washed three times in RPMI 1640 and seeded in U-shaped 96-well microtiter plates (Flow Laboratories) at 1×10⁴ cells/well. The effector cells suspension was added to quadruplicate wells to give three E/T ratios (i.e. 100:1, 50:1, 25:1) in a final volume of 200 μl per well. The plates were then incubated for 4 hours at 37° C. in a 5% CO₂ incubator, 100 μl of supernatants were collected from each well, and the radioactivity was measured using a gamma counter. Total mean cytotoxicity±S.E.M. were calculated from quadruplicate cpm values from individual spleens.

### Immunofluorescence Staining and Flow Cytometric Analysis of Spleen Cell Subsets

Splenocytes from individual mice were analysed by flow cytometry. The following monoclonal antibodies were used for double fluorescence analysis of spleen cell subsets: fluorescein (FITC)-conjugated anti-mouse NK1.1 PE (PharMingen, San Diego, Calif.), PE-conjugated anti-mouse CD4 (PharMingen), FITC-conjugated anti-mouse CD8 (PharMingen). Approximately 1×10⁶ spleen cells were resuspended in 50 ml of CM and staining was performed at 4° C. for 30 minutes. Cells were then washed twice in PBS containing 0.02% sodium azide and flow cytometric analysis was performed using a FACscan flow cytometer (Becton Dickinson).

Fluorescence data were collected using a 488 nm excitation wavelength from a 15 mW air-cooled argon-ion laser. Emission was collected through a 585/42 nm band pass filter. A minimum of 5,000 events were collected on each sample and acquired in list mode by a Hewlett Packard 9000 computer. To exclude dead cells, debris, non lymphoid cells, and cell aggregates, data collection was gated on live spleen lymphocytes by forward and side angle scatter. Data are represented as the percentage of positive cells over the total number of cells counted.

### Statistical Analysis

Kaplan-Meier method was used to estimate the survivor functions and Log-rank test was performed for testing the homogeneity of survival functions across the four groups (control, CY, OM-174, CY+OM-174).

Tumor growth was analyzed by T-test for unpaired data.

Student's T-test was employed to analyse mean control values in the other experiments. Values of less than 0.05 were considered significant.

### Results

### Tumour Growth

As shown in FIG. 1, both CY and OM-174, when used individually, inhibited slightly but significantly B16 tumour growth as compared to the untreated controls. Importantly, the combination OM-174 and CY leads to a better inhibition of tumour growth rate, which was significantly better than that obtained by means of the single treatments.

### Survival Time

Both CY and OM-174, when used alone, increased slightly but significantly the mean survival time (MST) of mice with respect to the untreated controls. The combined treatment with CY and OM-174, induced the better results in terms of survival of mice, which was significantly higher than that of control mice but also of mice receiving CY or OM-174 alone. FIG. 2 shows the percentage of animals surviving in each treatment group during the whole period of observation.

### NK Activity

Tumour cell elimination is known to be mediated in part by the cytotoxic activity of NK cells. It has been therefore measured the cytotoxic activity of splenocytes against NK-sensitive (YAC-1) tumour cells. Spleen cells were obtained from normal mice or from tumour-bearing mice that had been treated with PBS, CY, OM-174, or CY in combination with OM-174. Results are represented graphically in Table 2.

TABLE 2
Effect of treatment on NK and CTL activities

| Treatment | NK % cytotoxicity | CTL % cytotoxicity |
|---|---|---|
| group | (E/T ratio 25:1) | (E/T ratio 25:1) |
| Normal (N) | 3.73 ± 0.4 | <1 |
| N + OM-174 | 10.39 ± 1.0# | <1 |
| Tumour (T) | 2.69 ± 0.3 | 2.71 ± 0.3 |
| T + OM-174 | 6.3 ± 1.7 | 5.6 ± 0.9 |
| T + CY | 2.3 ± 0.4 | 2.63 ± 0.2 |
| T + CY + OM-174 | 12.4 ± 2.0* | 9.95 ± 1.6* |

On day 14 post-tumour injection, five mice per group were killed and cytotoxic NK and and CTL activities were measured as described in Materials and Methods. Results are expressed as mean percentage cytotoxicity ± S.E, derived from five individually tested mice per group.
#p < 0.001 vs. normal control mice./
*p < 001 vs. all the other groups of mice injected with B16 melanoma tumour.

In normal mice the treatment with OM-174 induced a dramatic increase of NK cell activity with respect to the untreated controls. The same dramatic increase of NK activity was observed also in B16 melanoma-injected mice. On day 14 both control and CY-treated tumour-bearing mice showed a decreased NK activity when compared to the untreated normal controls. OM-174 was always able to fully restore the NK activity over the levels observed in untreated normal controls. p<0.001 for T+CY+OM-174 vs. all other groups.

### Cytotoxic Activity Against Autologous Tumour Cells

Cytotoxic T lymphocytes (CTLs) also play an important role in the elimination of tumour cells. It has been tested from spleen cells from normal and tumour-bearing mice for specific cytotoxic activity against autologous tumour cells using in-vivo passaged B16 melanoma cells as target. The results of these experiments are shown in Table 2 above. As expected, it has been found that spleen cells from normal mice showed no detectable cytotoxic activity against B16 cells. On the contrary, splenocytes from tumour-bearing mice showed an appreciable cytotoxic activity against autologous tumour cells, which appeared not to be increased by CY treatment. The administration of OM-174 was capable of inducing a marked stimulation of CTL activity in tumour-bearing mice (two-fold increase). Interestingly, in mice treated with the combination of OM-174 and CY, the highest levels of cytotoxic activity against autologous tumour cells has been shown to be increased 4-fold with respect to those of tumour controls and 2-fold with respect to those of tumour mice treated with OM-174 alone.

### Analysis of Spleen Cell Subset

To assess the impact of the different treatments on lymphocyte subsets of the experimental mice and their correlation with the results obtained on tumour growth, survival time, and cytotoxic activities, the percentages of spleen cells expressing CD4, CD8, and NK1.1. have been measured.

As shown in Table 3, tumour-bearing mice showed a significant reduction in all the spleen cell subsets tested compared to normal controls. The treatment with OM-174 increased the percentages of CD4⁺, CD8⁺ and NK 1.1 positive cells both in normal and in tumour-bearing mice. As already mentioned for the other parameters analysed, the highest percentages of CD4⁺, CD8⁺ and NK1.1 positive cells were found in mice treated with CY+OM-174, which were over the values found in normal mice.

TABLE 3
Effect of treatment on spleen lymphocyte subsets (%).

| Treatment group | CD4⁺ (%) | CD8⁺ (%) | NK (%) |
|---|---|---|---|
| Normal (N) | 28.5 ± 3.1 | 10.2 ± 1.6 | 9.2 ± 1.7 |
| N + OM-174 | 34.0 ± 2.5 | 12.6 ± 1.5 | 11.6 ± 2.1 |
| Tumour (T) | 18.9 ± 1.4 | 6.3 ± 0.9 | 5.4 ± 0.7 |
| T ± OM-174 | 27.0 ± 2.0 | 9.3 ± 1.8 | 7.5 ± 0.5 |
| T + CY | 21.7 ± 1.8 | 6.4 ± 1.4 | 4 ± 0.5 |
| T + CY + OM-174 | 32.7 ± 2.2* | 15.8 ± 1.9* | 10.9 ± 1.1* |

On day 14 post-tumour injection mice were killed and cells obtained from individually processed spleens were stained with monoclonal antibodies for FACS analysis. Results are expressed as mean percentages of positive cells vs total spleen cells ± S.E.M derived from five individually tested mice.
*p < 005 vs. all the other groups of mice injected with B16 melanoma tumour.

### Conclusion

In conclusion the present protocol of combined treatment seems highly effective in the model of B-16 melanoma, ascertaining the efficacy of immunochemotherapeutic protocols with lipid-A derivatives. Indeed, the results obtained on the stimulation of cytotoxic activities (non specific NK and cancer specific CTL) of spleen cells and on the increase of NK, CD4⁺ and CD8⁺ phenotypes following treatment with OM-174, alone or in combination with CY, correlate with the delay in tumour growth and with the prolonged survival time.

Based on these results, triacylated diphosphorylated lipid A derivatives of structural formula (II) may thus be considered as candidates for association with chemotherapeutic regimens in the treatment of cancer at clinical level.

### Example 2

### Antitumor Activity of Intratumoral OM-174 Combined with Intraperitoneal Cyclophosphamide on Advanced PROb Subcutaneous Colon Tumors in BDIX Rats

Here it was studied in a colorectal model of cancer cells the effect of a combined sequential therapy using first the well-recognized chemotherapeutic drug cyclophosphamide, to reduce the tumor-induced immunosuppression, followed by unspecific intratumoral immunostimulation with the triacylated lipid-A derivative OM-174. In contrast to the results obtained with other immunostimulating drugs such as CpG or BCG, it is demonstrated here that the antitumoral activity of cyclophosphamide was highly increased when this standard treatment was followed by intratumoral injections of OM-174.

### Material, Methods and Statistics

### Animals

Female inbred BDIX-strain rats 4 to 6 months old, weighing 200-250 g, were bred in constant conditions of temperature, hygrometry and exposure to artificial light.

### Chemical and Drugs

OM-174, was from OM PHARMA, cyclophosphamide (CY) from Sigma-Aldrich (L'Isle d'Abeau, France), intradermic BCG (BCG Vaccine) from Pasteur Vaccines (Lyon, France). CpG (synthetic polynucleotides) was synthesized internally in the laboratory of Prof Chauffert (Dijon, France).

### Cancer Cells and Tumor Model

The DHD/K12 cells originated from a dimethylhydrazine-induced colon tumor in BD IX rats. The PROb clone was chosen for its regular tumorigenicity when injected into syngeneic rats. PROb cells were maintained in culture in Ham's F10 medium supplemented with 10% fetal bovine serum. Cells were detached with trypsin and EDTA and centrifuged in the presence of complete culture medium with fetal bovine serum to inhibit trypsin. Cells (2×10⁶/rat) were suspended in 0.1 ml of serum-free Ham's F10 medium then s.c. inoculated in the anterior thoracic area of anesthetized rats.

### Treatments of Animals

Female BDIX rats treatment started at day 36 after the s.c. inoculation of PROb cancer cells, when the tumor volume was about 1 cm³. Experiments consisted of 8 groups of rats (6 animals in each group). Control group received no treatment. The other groups received either an unique injection of CY by the i.p. route (25 mg/kg in 5 ml of a sterile NaCl solution), or immunostimulants by the intratumoral (i.t.) route starting at day 43, or i.p. CPM at day 36 combined with i.t. immunostimulant starting at day 43. i.t. Injections were done at day 43 and 50 for BCG (100 μl of the reconstituted solution+100 μl NaCl for every intratumoral injection). CpG (100 μg/injection in 200 μl NaCl) and OM-174 (200 μg/injection in 200 μl NaCl), were i.t. injected three times a week for 4 weeks (12 injections). Tumor diameter was measured once a week with a calliper.

### Results and Discussion

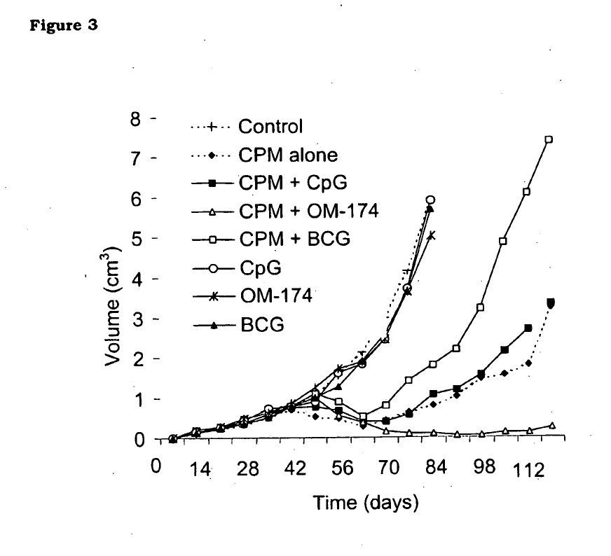

Intratumoral immunostimulants alone (OM-174, BCG, CpG) have no antitumoral effect comparatively to untreated animals on these large, established PROb tumors (FIG. 3). In contrast, i.p. cyclophosphamide caused a transient regression of the subcutaneous tumors, followed by a growth resumption in all animals. This was in accordance with the known chemosensitivity of the PROb cells to alkylating agents (Chauffert et al, 1992). However, CPM alone was unable to cure animals. BCG had a deleterious effect, since its association to CY was less active than CY alone. CpG did not modify the CY activity. In contrast to the other immunostimulants, OM-174 strongly enhanced the antitumor affect of CY. All tumors regressed at a greater extent than in animals treated with CY alone and a complete and lasting tumor regression was obtained in 4/6 animals in this group (see Table 4).

TABLE 4
Number of cured animals after various treatments

| | Number of cured animals/ |
|---|---|
| Treatment | total number of animals |
| Control | 0/6 |
| BCG | 0/5 |
| CpG | 0/6 |
| OM-174 | 1/6 |
| CY | 0/6 |
| CY + BCG | 1/6 |
| CY + CpG | 0/6 |
| CY + OM-174 | 4/6 |

In conclusion, these results demonstrate that OM-174 enhanced the antitumor effect of cyclophosphamide on advanced subcutaneous tumors in rats. In the present experiment, two other immunostimulants, BCG and CpG, worsened or did not improve at all the effect of cyclophosphamide alone, respectively.

### Example 3

### Enhancement of the Anticancer Effect of the Chemotherapeutic Agent Cisplatin in Combination with OM-174

### Introduction

It has been demonstrated many times in the past the antitumoral effect of the immunostimulating agent OM-174 in the BDIX/ProB model of peritoneal carcinomatoses in the rat (e.g. Onier et al., Clin Exp Metastasis. 1999 June; 17(4):299-306.). It has been shown that the beneficial effect is even maximal (90% of complete remissions) when the treatment starts 14 days after the injection of the cancer cells (syngenic Prob cells). In contrast, the efficacy of the product is diminished when the treatment starts on D21, or D28, and even disappears when treatment starts on D35. In order to find a therapy which could be adapted to humans, it has been tested here a combination of OM-174 with the platin oncostatic alkylating agent cisplatin, by selecting experimental conditions in which OM-174 per se is not optimally active. As it will be presented below, the results suggest that the combination cisplatin/OM-174 may have a therapeutic effect in humans, since when cisplatin (3 mg/kg, i.v.) is provided on D21, OM-174 is still highly effective, even when injected for the first time on D21 or D28, and even sometime on D35.

The following Procedure was Followed:

### Cancer Cells

Colon cancer PROb cells were originally obtained from a tumor of a BDIX rat induced by 1,2-dimethylhydrazine.

The BDIX strain of rats was established in 1937 by H. Druckrey. Nowadays these rats come from Iffa-Credo (L'Asbresle, France).

BDIX rats, 4 months±1 month at the beginning of the experiment, 7 animals/group, received i.p. cultured syngenic PROb cells (i.p) on day 0. Cisplatin (3 mg/kg) was injected i.v. on day 21, and OM-174 treatment (1 mg/kg, 5 injections i.v. in the penile vein every 5th day) started either on days 28 or 35. Survival was followed until day 72 in the example presented here.

### Results

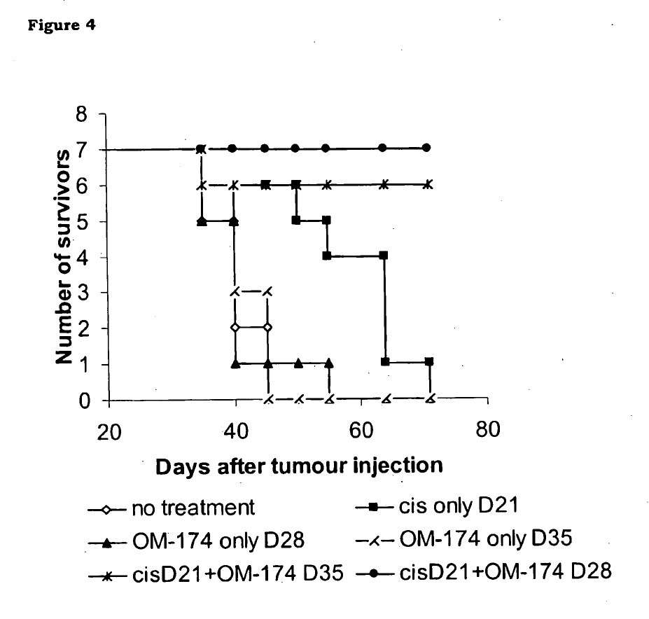

OM-174 per se is fully able to display anticancer effects when treatment (1 mg/kg, up to 15 injections i.v. every 2nd day) starts until 2 weeks after tumour inoculation. However the anticancer effect is lost when treatment is started later (day 28 or day 35 as shown in FIG. 4). This less favourable condition is certainly closer to the real clinical situation encountered in many cancerous patients.

In this example, cisplatin (3 mg/kg i.v) is given on day 21. A further immunostimulating treatment with OM-174 is started only on day 28 or 35 (1 mg/kg, 5 injections i.v. every 5^{th} day). The survival curves are shown in FIG. 4.

### Conclusion

The combination of OM-174 treatment with cisplatin, in this very unfavorable environment, gave a much stronger antitumour activity than either treatment alone.

Cisplatin treatment, as shown here, displays only partial efficacy, but when boosted by OM-174 immunostimulation, it reveals a strong antitumour effect.

### Example 4

### Enhancement of the Anticancer Effect of the Chemotherapeutic Agent 5-Fluouracil (5-FU) in Combination with OM-174

### Introduction

Antimetabolites interfere with normal metabolic pathways, including those necessary for making new DNA (phase S of the cell cycle). This class of molecules is often used to treat cancer.

A clinically efficient antimetabolite drug that disturbs DNA synthesis is 5-FU, used since at least four decades (see e.g Rich et al., 2004). It has a wide range of activity including colon cancer, breast cancer, head and neck cancer, pancreatic cancer, gastric cancer, anal cancer, oesophageal cancer and hepatomas.

An adequate and timely controlled clinical combined therapy with a well-recognized chemotherapeutic drug such as 5-FU, used first to shrink and kill some cancer cells (and thus potentially reveal tumor-associated antigens), followed by an unspecific immunostimulation with triacylated lipid-A derivatives will probably enhance the efficacy of the oncostatic drug, and permits the acquisition of an immunological (specific) memory to get rid of cells bearing the tumor associated antigen, and also to limit the level of the side effects observed, by allowing e.g. to reduce the number of administrations and/or the doses of the chemotherapeutic drug.

This experiment was aimed to check the efficacy of the combination of 5-FU with OM-174 in a rat model of colon cancer.

### Material and Methods

The following procedure was followed:

The products: OM-174-DP was tested in association or not with 5-FU as described below:

On day 0 (D0), 106 PROb cells were injected i.p. to each rat. 5-FU was administered i.p. at the dose of 30 mg/kg on days 7 and 14. OM-174 was injected at the dose of 1 mg/kg i.v. from day 21 three times a week for a total of 10 injections.

### Readouts

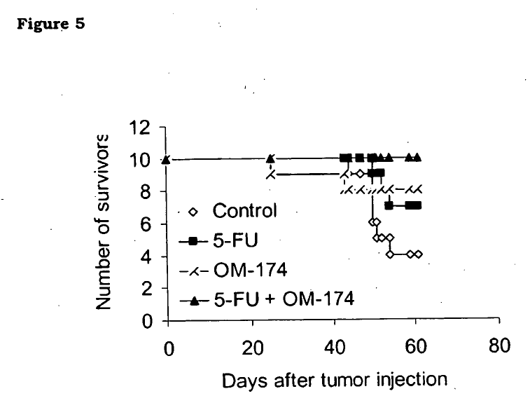

All rats (controls and treated) were sacrificed by CO₂ on day 61. The efficacy of the treatment was determined by read-outs such as survival (FIG. 5) and measure of the classes of cancer given depending on the number and the size of the nodules, and also by ascites measurements. Carcinomatoses were evaluated blindly. As it is impossible to measure the volume of a carcinomatosis, they were classified according to the number and diameter of the nodules:
Class 0: no visible nodule
Class 1: some countable nodules with a diameter from 0.1 to 0.3 cm
Class 2: many uncountable nodules with a diameter from 0.1 to 0.3 cm
Class 3: some nodules with a diameter of 1 cm invade the peritoneal cavity
Class 4: the cavity is completely invaded by tumor masses of several cm.

The ascite volume was measured by double weights of the rats.

### Results:

see the Table 5 and FIG. 5:

TABLE 5
carciomatosis classes and ascites volumes after treatments

| | Number of rats in each | | | | | |
|---|---|---|---|---|---|---|
| | carcinomatosis classes | | | | | |
| Groups/read- | (0, 1, 2, 3, and 4) | | | | | |
| outs | 0 | 1 | 2 | 3 | 4 | Ascites (ml) |
| Control | 0 | 0 | 1 | 0 | 8 | 57 |
| 5-FU | 2 | 0 | 0 | 0 | 8 | 44 |
| OM-174 | 2 | 3 | 2 | 2 | 1 | 1 |
| OM-174 + 5-FU | 8 | 0 | 0 | 0 | 1 | 0 |

### Concerning the Classes:

The Mann-Whitney test shows a significant difference between Control and OM-174 groups as well as between Control and 5-FU+OM-174 groups. No significant difference has been shown for 5-FU versus Control groups. There is a significant difference in the median scores between the Control group and both the OM-174-DP and the 5-FU+OM-174-DP groups (DP means diphosphorylated derivative).

The corresponding survival curve is shown on FIG. 5.

### Conclusion

The combination OM-174+5-FU is better in term of carcinomatosis classes and survival time than both agents taken individually in this model of cancer.

### Example 5

### OM-174 in Combination in Radiotherapy

Solid tumors are supplied with lower oxygen levels than normal tissues because of poorly developed vasculature and sporadic occlusion of blood vessels (van der Berge et al., 2001). Hypoxia-induced radioresistance is recognized as a major obstacle in the treatment of cancer (Dachs and Stratford, 1996). The possibility to radiosensitize hypoxic tumor cells by an immunostimulating agent able to induce nitric oxide radical (NO, a gas fixing the DNA damage caused by radiation) is presented below. It will be shown that OM-174-induced NO appears to be a potent radiosensitizer in mouse EMT-6 tumor cells, both directly in hypoxic conditions, and also indirectly via activation of cytokines released by macrophages.

### A) Direct Effect of OM-174 on EMT-6 Breast Cancer Cells.

The direct radioprotective effect of OM-174 on the cancer cells EMT-6 was tested first in vitro both in normal (21%) and hypoxic (1%) oxygen conditions. The hypoxic condition really reflects the situation of cancer cells located from a few micrometers away from a capillary. To get rid of these cells, higher doses of radiation are required, therefore agents such as OM-174, either injected intratumorally, or i.v. may be of interest.

Murine mammary adenocarcinoma EMT-6 cells were cultured in RPMI medium+10% bovine calf serum in plastic flasks. EMT-6 monolayer cultures grown to early confluence were exposed to OM-174 for 16 hours in both conditions (21% and 1% oxygen). After treatment with OM-174, nitrite determination using the classical Griess method was performed. Values were normalized for 200'000 cells per well.

Cells were then collected by trypsinization and the radioresponse was estimated as described previously (Van der Berge et al., 2001) Briefly, micropellets (0.5×10⁶ cells) were produced in conical tubes by centrifugation at 300 g for 5 min. Metabolic oxygen depletion in micropellets was induced by incubation at 37° C. for 3 minutes prior to radiation. Micropellets were irradiated with a linear accelerator at a rate of 2 Gy per min and the survival fraction (SF) after 5, 10, 15, and 20 Gy was measured by a 8-day colony formation assay.

### Results

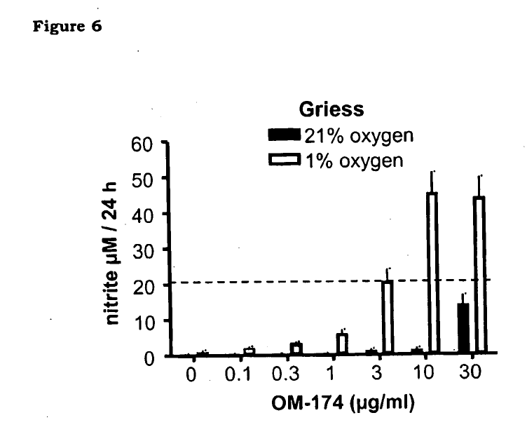

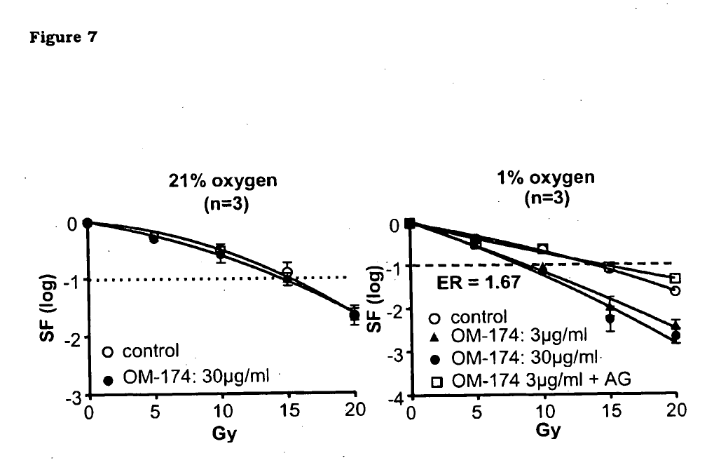

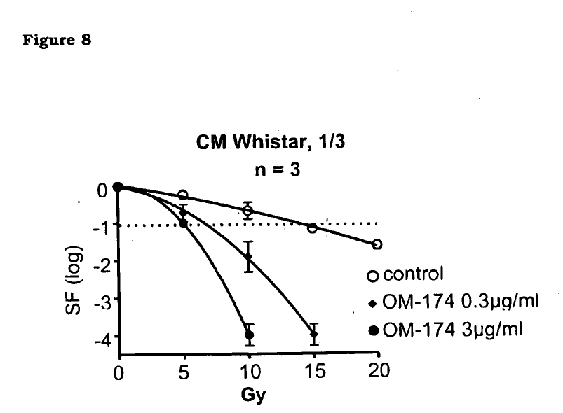

As shown in FIG. 6, EMT-6 cells produced low amounts of NO when stimulated by OM-174 in normal oxygen levels (21% oxygen). In contrast, an increased production of NO was detected in hypoxic (1% oxygen) condition. Interestingly, the direct clonogenic assay (FIG. 7) shows that OM-174 is a directly radiosensibilizing agent for cancer cells only in hypoxic conditions (the radiation dose necessary to kill 90% of the cells was 1.67 lower than in the absence of OM-174) (at either 3 or 30 mg/ml). The indirect radiosensibilizing effect via OM-174-induced conditioned medium (CM) from Whistar rats is shown in FIG. 8. In these conditions, the higher dose tested (3 μg/ml) was clearly more radiosensibilizing than the dose of 0.3 μg/ml.

### Conclusion

These results suggest that OM-174 displays both direct and indirect radiosensibilizing properties and therefore triacylated diphosphorylated lipid-A derivatives of structural formula (II) are good candidates to be combined with radiotherapy.

### GENERAL CONCLUSION

In summary these results appear promising and suggest that non-specific immuno-stimulation by triacylated diphosphorylated lipid-A derivatives of structural formula (II), and particularly the well tolerated compound OM-174 have strong potential to improve the anticancer effects obtained by well-established or experimental anticancer therapies, particularly classical chemotherapy and radiotherapy.

Immunotherapy with a triacylated diphosphorylated lipid-A derivative of structural formula (II) in any appropriate formulation, dose, frequency of administration will be applied in humans repeatedly parenterally, preferentially by the intravenous or intratumoral routes. The preferred treatment selected from chemotherapy and/or radiotherapy will be applied each time according to standard practice (formulation, dose, frequency and route), either before, concomitantly, or after immunotherapy.

The needed dosages of a compound of formula II will range from 0.05 to 100 mg/m² for the humans and preferably from 0.1 to 20 mg/m².

The antineoplastic agent is used at doses very broadly ranging from 0.1 to 200 mg/kg.

What is claimed is:

1) A pharmaceutical composition intended for treating warm-blooded animals including humans, suffering from a proliferative disease, comprising as the active ingredient a non specific immuno modulating agent compound with charged or neutral groups of general formula I: 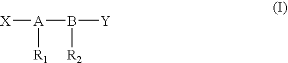
wherein
A-B is a disaccharide structure,
X and Y are charged or neutral functional groups and
R₁ and R₂ are hydroxyacyl groups which may be acylated with an aliphatic acid,
together with a known antineoplastic agent selected from the group consisting of radiation therapy and one or more chemotherapeutic agent selected from the group consisting of alkylating agents, antimetabolites, agents acting on tubules, tyrosine-kinase inhibitors,
in conjugation or admixture with an inert non-toxic pharmaceutically acceptable diluent or carrier

2) A pharmaceutical composition according to claim 1,
wherein the diacylated compound with charged or neutral groups of general formula I is in the form of a mineral or organic base addition salt.

3) A pharmaceutical composition according to claim 1,
wherein the diacylated compound is esterified with a hydroxy dodecanoyl moiety.

4) A pharmaceutical composition according to claim 1,
wherein the antineoplastic agent is in the form of a pharmaceutically acceptable addition salt.

5) A pharmaceutical composition according to any of claims 1 to 4,
in which the active ingredient is a diacylated compound with charged or neutral groups of general formula I: 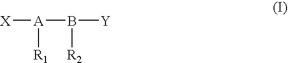
wherein
A-B is the central β(1,6) linked diglucosamine disaccharide back bone of lipid A
of formula II: 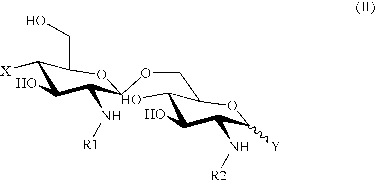
wherein R₁ and R₂, each designate an acyl group derived from a saturated or unsaturated, straight or branched-chain carboxylic acid having from 2 to 24 carbon atoms, which is unsubstituted or bears one or more substituents selected among hydroxyl, alkyl, alkoxy, acyloxy, amino, acylamino, acylthio and alkylthio groups,
X designates a neutral or charged group selected among the following groups: dihydroxyphosphoryloxy, hydroxysulfonyloxy, hydroxyle, carboxyalkoxy, carboxyalkylthio, carboxyacyloxy, carboxyaminoacyloxy, or diaminoacyloxy, aminoacyloxy
Y designates a neutral or charged group selected among the following groups: dihydroxyphosphoryloxy, hydroxysulfonyloxy, hydroxyle, carboxyalkoxy, carboxyalkylthio, carboxyaminoalkoxy, aminoalkoxy and the waved line an α or β orientation.

6) A pharmaceutical composition in accordance with claim 1,
wherein the active ingredient is a triacylated diphosphorylated lipid-A derivatives of structural formula (III)

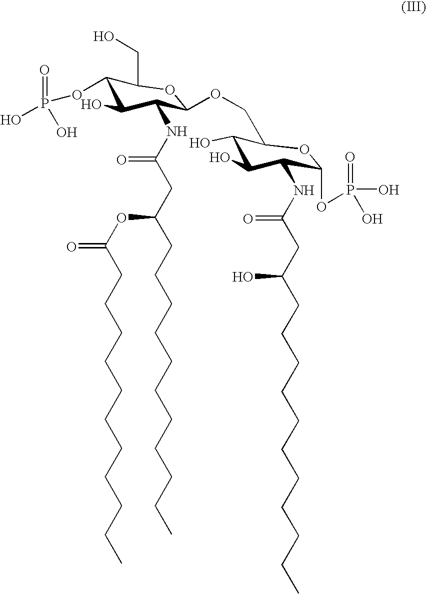

7) A pharmaceutical composition according to claim 1,
wherein the antineoplastic means belongs to one of the following classes:
(a) chemotherapeutic drugs,
(b) ionising radiation

8) A pharmaceutical composition according to claim 6,
wherein the chemotherapeutic drug is an alkylating agent or an antimetabolite agent.

9) A pharmaceutical composition according to claim 6,
wherein the alkylating agent is selected from the group consisting of carboplatin, cisplatin, oxaliplatin, cyclophosphamide.

10) A pharmaceutical composition according to any of the preceding claims,
wherein the antimetabolites are selected from the group consisting of antifolic agents, purine analogs, and pyrimidine analogs.

11) A pharmaceutical composition according to claim 9,
wherein the purine and pyrimidine analogs are selected from the group consisting of Capecitabine, Cladribine, Cytarabine, Fludarabine, Fluoro uracile (5-FU), Gemcitabine, Mercaptopurine, Methotrexate, Thioguanine and the like.

12) A pharmaceutical composition according to claim 6,
wherein the treatment with the immunostimulating agent according to claim 1 is accompanied or followed with a well-known or experimental anticancer ionising radiation therapy.

13) A process for treating cancer in warm-blooded animal including humans,
which consists in administering a combination of a therapeutically effective amount of a mixture of compounds according to claim 1 in a pharmaceutically-acceptable carrier, excipient or formulation via the oral, parenteral, rectal, topical, subcutaneous or submucosal route.

14) The method of claim 13,
wherein the active ingredient which is a triacylated compound with charged or neutral groups of general formula I and wherein the well-established or experimental anticancer treatment, which belongs to the following classes:
(a) chemotherapeutic drugs,
(b) ionizing radiation,
are simultaneously administered.

15) The method of claim 13,
wherein the pharmaceutical compound with charged or neutral groups of general formula I and the well-established or experimental anticancer treatment, belonging to the following classes:
(a) chemotherapeutic drugs,
(b) ionizing radiation
are sequentially administered.

16) The method of claim 13,
wherein the pharmaceutical compound with charged or neutral groups of general formula I and the well-established or experimental anticancer treatment, belonging to the following classes:
(a) chemotherapeutic drugs,
(b) ionising radiation,
are applied locally to the cancer tissue.

17) The method of claim 13,
wherein the a pharmaceutical compound with charged or neutral groups of general formula I and the well-established or experimental anticancer treatment belonging to the following classes:
(a) chemotherapeutic drugs,
(b) ionizing radiation,
are applied in a controlled or sustained delivery carrier.

18) The method of treatment according to claims 13 to 17,
where the proliferative cancerous disorder belongs to the groups of lung cancer, breast cancer, colorectal cancer, stomach cancer, liver cancer, cervical cancer, esophageal cancer, head and neck cancer, bladder cancer, malignant non-Hodgkin lymphomas, leukaemia, prostate cancer and testicular cancer, pancreatic cancer, ovarian cancer, kidney cancer, endometrial cancer, nervous system cancer, melanoma, thyroid cancer, pharynx cancer and Hodgkin disease cancer, squamous cell carcinoma, glioma, myeloma and other solid or lymphoma cancers.

19) A method according to claim 13,
wherein the anticancer agent is selected from platin complexes such as carboplatin and oxaliplatin and the proliferative cancerous disorder is testicular, bladder, lung, gullet, stomach, colorectal and ovarian cancers.

20) A method according to claim 13,
wherein the anticancer agent is selected among cyclophosphamide derivatives where the proliferative disorder is chronic lymphocytic leukaemia, lymphomas, ovarian cancer, and bladder cancer.

21) A method according to claim 13,
wherein said anticancer agent is 5-fluorouracil 5-FU and 5-FU derivatives, and the proliferative disorder is bowel, breast, stomach, and gullet cancer.

22) A pharmaceutical composition according to claim 1,
wherein the active compound or a salt of the compound according to formula (I) and the antineoplastic agent are present within a single container.

23) A pharmaceutical composition according to claim 1,
wherein the compound or a salt of the compound according to formula (I) and the antineoplastic agent are disposed within distinct containers.
